(19) United States
(12) Patent Application Publication
(10) Pub. No.: US 2019/0060620 A1
(43) Pub. Date: Feb. 28, 2019

(54) INTEGRATED STENT AND DELIVERY SYSTEM

(71) Applicant: Boston Scientific Scimed, Inc., Maple Grove, MN (US)

(72) Inventors: Jeff Gray, Lexington, MA (US); Scott E. Brechbiel, Acton, MA (US); Paul Smith, Smithfield, RI (US); Laura E. Christakis, Framingham, MA (US); Ryan V. Wales, Northborough, MA (US); Sean P. Fleury, Minneapolis, MN (US); Bryan Bannon, Duxbury, MA (US); Anand V. Sahai, Montreal (CA)

(21) Appl. No.: 16/115,936

(22) Filed: Aug. 29, 2018

Related U.S. Application Data
(60) Provisional application No. 62/552,227, filed on Aug. 30, 2017.

(51) Int. Cl. A61M 27/00

(52) U.S. Cl. CPC A61M 27/002; A61M 2210/1082; A61M 2210/1085; A61B 18/14; A61M 2210/1075; A61M 2205/0266; A61M 2210/1057; A61M 2210/1053

(57) ABSTRACT

The present disclosure relates generally to the field of medical devices and establishing fluid communication between body lumens and/or within a body vessel. In particular, the present disclosure relates to devices and methods for establishing drainage between apposed body lumens and/or within a body vessel.

### CROSS REFERENCE TO RELATED APPLICATIONS

This application claims the benefit of priority under 35 U.S.C. § 119 to U.S. Provisional Patent Application Ser. No. 62/552,227, filed on Aug. 30, 2017, which is incorporated by reference in its entirety for all purposes.

### FIELD

The present disclosure relates generally to the field of medical devices and establishing fluid communication between body lumens and/or within a body vessel. In particular, the present disclosure relates to devices and methods for establishing drainage between apposed body lumens and/or within a body vessel.

### BACKGROUND

As an example of medical devices for establishing fluid communication, stent delivery systems, including the Hot Axios™ Stent and Electrocautery-Enhanced Delivery System (Boston Scientific Corp., Marlborough Mass.), allow drainage of fluid collections, such as pancreatic pseudocysts. A significant advantage of these all-in-one stent delivery systems is their convenience and ease of use, which in the case of the Hot Axios system allow the “hot” electrocautery-enhanced catheter tip to access a target body lumen and deliver the stent without having to introduce a separate stent delivery system. The ability to access a target area and deploy a stent directly from the electrocautery system is advantageous in terms of procedure times, safety and accuracy of stent placement, since the physician may avoid multiple device exchanges and the adherent risk of losing proper endoscope position while manipulating the various tools such as guidewires, needles and delivery systems that may be required for a given procedure. Due to this convenience, similar delivery systems might be beneficial in situations in which a different configuration of stent may be more appropriate. For example, a patient may present with a non-advanced disease state in which the collection to be drained is truly fluid filled, e.g., without the presence of solid or semi-solid material which require the larger drainage lumen (10 mm or more) of the Axios™ stent.

A variety of advantageous medical outcomes may therefore be realized by the devices and/or methods of the present disclosure, which provide convenient and efficient stent delivery systems for different configurations of drainage stents, including low-profile plastic stents, among other embodiments.

### SUMMARY

In one aspect, the present disclosure relates to a medical device comprising a delivery tube with a proximal end, a distal end, and a lumen extending between the proximal and distal ends. An actuation member may extend through the lumen of the delivery tube. A distal end of the actuation member may be attached to the distal end of the delivery tube. A first elongate tubular body may be disposed around a distal portion of the delivery tube. The first elongate tubular body may comprise a proximal end, a distal end, and a lumen may extend between the proximal and distal ends. A slit may extend along an entire length of the first elongate tubular body. The distal portion of the delivery tube may be configured to move from a first position to an expanded second position when the actuation member is proximally retracted. A portion of the first elongate tubular body may be deployable from around the distal portion of the delivery tube when the delivery tube moves from the first position to the second position. The distal portion of the delivery tube may be extendable through the slit of the first elongate tubular body when in the second position. The distal portion of the delivery tube may form into a malecot configuration when in the second position. A second elongate tubular body may be disposed around the first elongate tubular body. The second elongate tubular body may comprise a proximal end, a distal end, and a lumen may extend between the proximal and distal ends. A slit may extend along an entire length of the second elongate tubular body. The first elongate tubular body may include a plastic or polymeric stent.

In another aspect, the present disclosure relates to a medical device comprising a sheath. A pusher member may be slidably disposed within a portion of the sheath. A delivery tube may be slidably disposed within the sheath. A proximal end of the delivery tube may be attached to a distal end of the pusher member. A first elongate tubular body may be disposed about a distal portion of the delivery tube. A second elongate tubular body may be disposed about a proximal portion of the delivery tube. Each of the first and second elongate tubular bodies may include a proximal end, a distal end and a slit extending along a length thereof. One or more projections may be disposed within the distal portion of the delivery tube. The one or more projections may be configured to move from a first position to a second position. The one or more projections may be retractable within the distal portion of the delivery tube when in the first position. The one or more projections may be extendable laterally outward from the distal portion of the delivery tube when in the second position. The one or more projections may deploy the first elongate tubular body from about the distal portion of the delivery tube when in the second position. The pusher member may be configured to advance the second elongate tubular body to the distal portion of the delivery tube after the first elongate tubular body is deployed. The one or more projections may deploy the second elongate tubular body from about the distal portion of the delivery tube when in the second position.

In another aspect, the present disclosure relates to a medical device comprising an outer sheath. A pusher member may be slidably disposed within a portion of the outer sheath. A delivery tube may be slidably disposed within the outer sheath. A proximal end of the delivery tube may be attached to a distal end of the pusher member. A first elongate tubular body may be disposed about a distal portion of the delivery tube. A second elongate tubular body may be disposed about a proximal portion of the delivery tube. Each of the first and second elongate tubular bodies may include a proximal end, a distal end and a slit may extend along a length thereof. An inner sheath may be slidably disposed within the outer sheath. A first portion of the inner sheath may be disposed about the pusher member and the second elongate tubular body. A second portion of the inner sheath may be disposed within a lumen of the first elongate tubular body. A shape memory wire may be embedded within the inner sheath. A portion of the inner sheath may be configured to move between a first configuration and a second configuration to deploy the first elongate tubular body. The pusher member may be configured to advance and deploy the second elongate tubular body. The inner sheath and outer sheath may be retractable from the second elongate tubular body.

In another aspect, the present disclosure relates to a medical device comprising a delivery tube with a proximal end and a distal end. A lumen may extend between the proximal and distal ends. An elongate tubular body may be disposed around a distal portion of the delivery tube. The elongate tubular body may comprise a proximal end, a distal end, and a lumen may extend between the proximal and distal ends. A conical cap may be attached to a distal end of the delivery tube. A distal end of the elongate tubular body may include a chevron shape configured to be received within a corresponding recess formed within the conical cap. The delivery tube may be proximally retracted such that the distal end of the elongate tubular body may ride up and over the recess of the conical cap, to separate or peel the elongate tubular body from around the delivery tube.

In another aspect, the present disclosure relates to a medical device comprising a delivery tube with a proximal end and a distal end. A lumen may extend between the proximal and distal ends. A number of elongate tubular bodies (e.g., four elongate tubular bodies) may be disposed around a distal portion of the delivery tube. The elongate tubular bodies may comprise a proximal end, a distal end, and a lumen may extend between the proximal and distal ends. A conical cap may be attached to a distal end of the delivery tube. A distal portion of the conical cap may include a series of projections. Each projection may be configured to align with a corresponding slit extending along a length of a respective one of the elongate tubular bodies. The delivery tube may be proximally retracted to draw each projection through the corresponding slit of each elongate tubular body to simultaneously separate or peel each elongate tubular body from around the delivery tube. A distal end of each elongate tubular body may include a surface feature (e.g., protrusion, enlarged portion, etc.) to provide a space between the outer surfaces of each elongate tubular body, and to guide the projections into the corresponding slits. An elongate tubular sheath may be disposed around the outermost tubular body, e.g., to maintain a proper position of the elongate tubular bodies on the delivery tube. A pusher member may be included about the delivery tube and proximal to the elongate tubular bodies. The pusher member may provide a backstop to the elongate body or bodies as the delivery tube is pulled proximally.

In another aspect, the present disclosure relates to a medical device comprising an elongate tubular body comprising a proximal end and a distal end. A lumen may extend between the proximal and distal ends. A slit may extend through a wall of the elongate tubular body along an entire length of one side of the elongate tubular body. A perforation or score-line may extend along an entire length of another side of the elongate tubular body opposite the slit. One or more openings may extend through the wall of the elongate tubular body. One or more retention members may be disposed along a portion of the elongate body. One edge of the elongate tubular body defined by the slit may be radially coiled within the lumen, and the other edge of the elongate tubular body defined by the slit may be disposed about an outer surface of the elongate tubular body. A dilation member (e.g., balloon, malecot, structural spreader, etc.) may be positioned within the lumen of the elongate tubular body. The dilation member may move from a first (e.g., non-expanded) configuration to a second (e.g., expanded) configuration with sufficient outward force to open the radially coiled elongate tubular body, and split the wall along the score-line. The dilation member may be attached to the distal end of an elongate member extending through the lumen of the elongate tubular body. The elongate member may be proximally retracted to draw the expanded dilation member through the lumen to separate the elongate tubular body along the entire length of the score-line to form two separate elongate tubular bodies.

In another aspect, the present disclosure relates to a medical device comprising a first elongate member with a proximal end and a distal end, and a second elongate member with a proximal end and a distal end. The distal end of the first elongate member may be attached to a first member of a delivery tip. The distal end of the second elongate member may be attached to a second member of the delivery tip. A first elongate tubular body (e.g., a self-expanding metallic stent, etc.) may be disposed about a distal portion of the first elongate member in a radially constrained configuration. A second elongate tubular body (e.g., a self-expanding metallic stent, etc.) may be disposed about a distal portion of the second elongate member in a radially constrained configuration. A surface of the first member may include a ridge or protrusion extending along a length thereof. A surface of the second member may include a corresponding recess or groove extending along a length thereof and configured to slidably receive the protrusion. The protrusion and recess may form a friction or interference fit to maintain the first and second members in a first configuration. The friction or interference fit may be sufficiently weak to allow the first and second members to be separated from each other, e.g., by proximally retracting the first elongate member and/or proximally advancing the second elongate member relative to the other. A distal end of the first member may include an edge or stop configured to contact a distal end of the second member, e.g., to prevent the second member from moving/sliding distally beyond the first member.

In another aspect, the present disclosure relates to a medical device comprising a first elongate member with a proximal end and a distal end, and a second elongate member with a proximal end and a distal end. The distal ends of the first and second elongate members may be reversibly attached to a delivery tip. The distal ends may include a frangible, or electrically erodible, material configured to break, separate or degrade upon receiving an appropriate signal or retractive force. A first expandable elongate tubular body (e.g., a self-expanding metallic stent, etc.) may be disposed about a distal portion of the first elongate member in a constrained configuration. A second elongate tubular body (e.g., a self-expanding metallic stent, etc.) may be disposed about a distal portion of the second elongate member in a constrained configuration.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIGS. 1A-1D provide perspective views of a medical device, according to one embodiment of the present disclosure.

FIGS. 2A-2B provide perspective views of a medical device, according to one embodiment of the present disclosure.

FIGS. 3A-3C provide perspective views of a medical device, according to one embodiment of the present disclosure.

FIGS. 4A-4B provide perspective views of a medical device, according to one embodiment of the present disclosure.

FIGS. 5A-5E provide perspective views of a medical device, according to one embodiment of the present disclosure.

FIGS. 6A-6B provide perspective views of a medical device, according to one embodiment of the present disclosure.

FIGS. 7A-7I depict exemplary steps involved in deploying a medical device, according to one embodiment of the present disclosure.

FIGS. 8A-8G depict exemplary steps involved in deploying a medical device, according to one embodiment of the present disclosure.

### DETAILED DESCRIPTION

The present disclosure is not limited to the particular embodiments described herein. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to medical devices (e.g., stents, etc.) and methods for drainage, it should be appreciated that such devices and methods may be used in a variety of medical procedures to establish or maintain a temporary or permanent open flow or access passage between or along a variety of lumens, vessels and/or apposed body organs, ducts, fistulas, cysts and spaces (e.g., the dermis, stomach, duodenum, jejunum, small intestine, gallbladder, kidneys, pancreas, biliary/pancreatic trees, bladder, ureter, walled-off pancreatic necrosis, abscesses, etc.). Moreover, such medical devices are not limited to single deployment, but may include multiple medical devices deployed simultaneously or sequentially.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

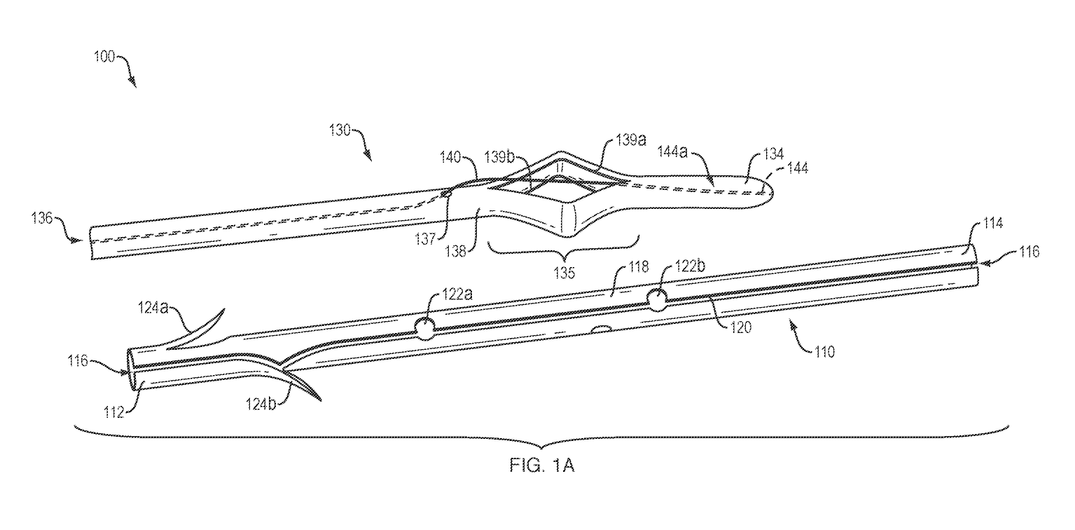

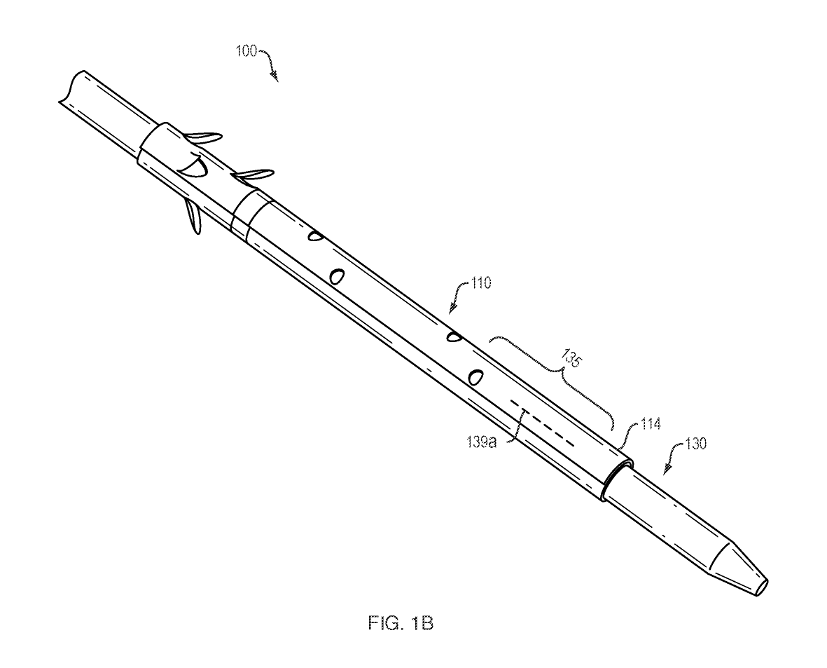

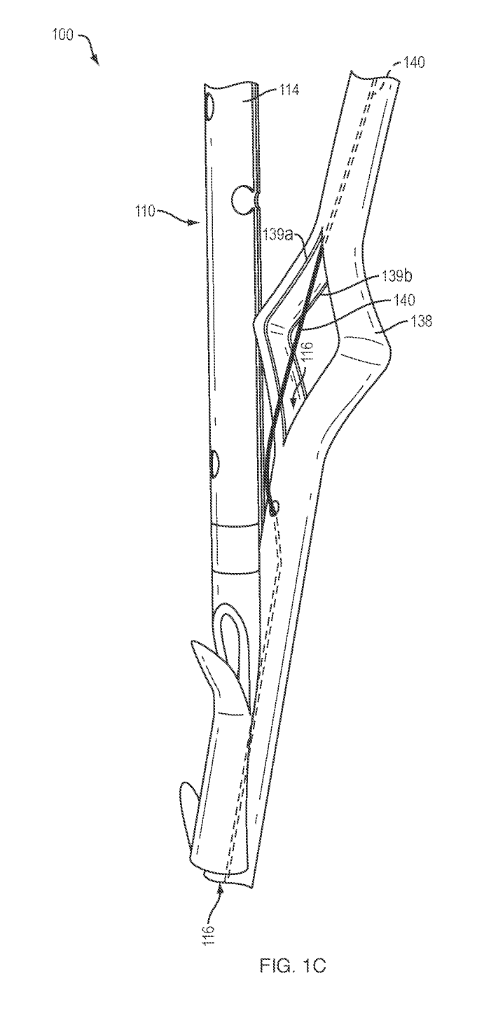

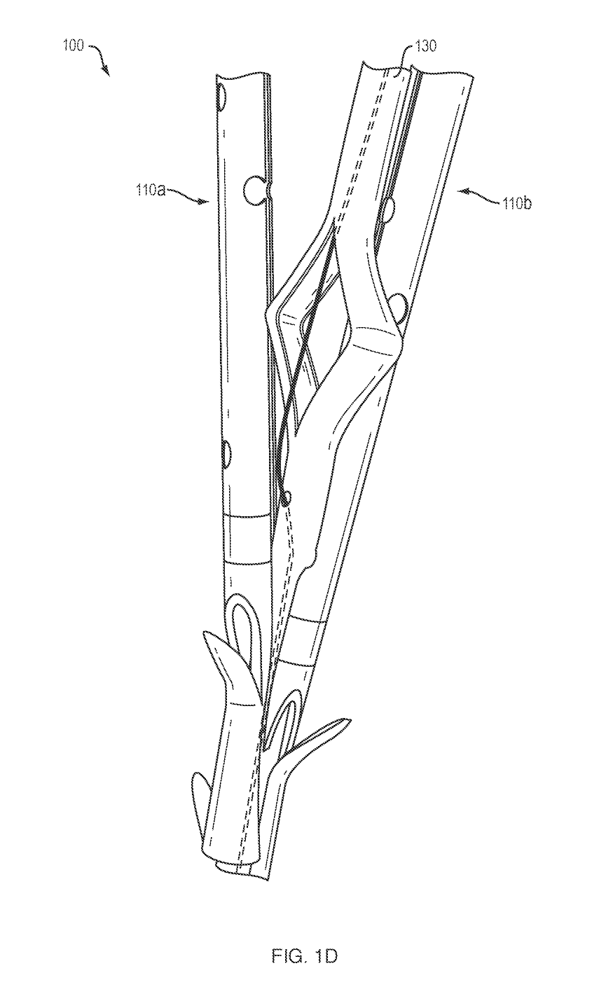

In various embodiments, the present disclosure relates to devices and methods for creating an open flow passage between apposed body lumens and/or within a body vessel. Referring to FIG. 1A, in one embodiment, a medical device 100 of the present disclosure may include an elongate tubular body 110 comprising a proximal end 112, a distal end 114 and a lumen 116 extending therebetween. A slit 120 may extend through a wall 118 of the elongate tubular body 110 along an entire length thereof. The medical device 100 may further include one or more openings 122a, 122b (e.g., to provide drainage from the exterior of the device to the lumen 116 extending along the interior), extending through the wall 118 of the elongate tubular body 110 and/or one or more retention members 124a, 124b disposed along a portion of the elongate tubular body 110.

Although the slit 120 is depicted as extending along a substantially straight or linear path relative to a longitudinal axis of the elongate tubular body 110, in various embodiments, the slit 120 may follow or include a variety of patterns (e.g., spiral, helical, etc.). In addition, the one or more openings 122a, 122b may be arranged in a variety of patterns about and/or along the elongate tubular body 110, some of which may intersect with the slit 120, and some of which may not intersect with the slit 120. In addition, the retention members 124a, 124b may be different in shape, number and configuration, and shapes and disposed at or near the proximal 112 and/or distal 114 ends of the elongate tubular body 110 and/or various locations therebetween in a variety of patterns, orientations and/or positions relative to a longitudinal axis of the medical device 100. In various other embodiments, the proximal and/or distal portions of the elongate tubular body 110 may be configured to form a coil or pigtail configuration when released from constraint, e.g., from within a delivery sheath or deployed from a delivery tube, discussed below.

In an embodiment, the medical device 100 may further include a delivery tube 130 sized and configured to be disposed within the lumen 116 of the elongate tubular body 110 (FIG. 1B). The delivery tube 130 may include a proximal end (not shown), a distal end 134 and a lumen 136 extending therebetween. A pair of slits 139a, 139b may extend through opposite sides of a wall 138 along a distal portion 135 of the delivery tube 130. An elongate actuation member 140 (e.g., suture, filament, etc.) may be disposed within at least a portion of the lumen 136 of the delivery tube 130. For example, in one embodiment, a distal portion 144a of the elongate actuation member 140 may extend within the lumen 136 of the delivery tube 130 distally beyond the slits 139a, 139b, and the distal end 144 of the elongate actuation member 140 may be secured or attached (e.g., integrally formed or molded, or adhered by using a suitable solder, glue, epoxy, resin, etc.) at or near the distal end 134 of the delivery tube 130. The elongate actuation member 140 may exit the lumen 136 through one of the slits 139a or 139b, and re-enter the lumen 136 through an opening 137 formed within the wall 138 of the delivery tube 130 and extend through a full length of the lumen 136. Alternatively, the elongate actuation member may extend within the lumen from the distal attachment point to its proximal end. Proximally retracting a proximal end (not shown) of the elongate actuation member 140 may urge a portion of the wall 138 of the delivery tube 130 co-extensive with the slits 139a, 139b to move from a first (e.g., linear, non-expanded, non-deployed etc.) configuration to a second (e.g., non-linear, expanded, deployed, etc.) configuration.

Referring to FIGS. 1B-1C, the elongate tubular body 110 may be disposed around a distal portion of the delivery tube 130 such that the slits 139a, 139b (not shown) are positioned at or near the distal end 114 of the elongate tubular body 110 (FIG. 1B). The proximal end (not shown) of the elongate actuation member 140 may be proximally retracted with sufficient force to urge the distal portion 135 of the wall 138 co-extensive with the slits 139a, 139b to move to the second configuration and laterally deploy a distal portion of the elongate tubular body 110 (FIG. 1C). The remaining portion of the elongate tubular body 110 may then be deployed by proximally retracting the delivery tube 130 and elongate actuation member 140 such that the expanded portion (e.g., malecot) of the delivery tube 130 is drawn through the lumen 116 to peel the elongate tubular body 110 from around or about the delivery tube 130.

Referring to FIG. 1D, in various embodiments, more than one elongate tubular body may be loaded onto the delivery tube 130 for simultaneous deployment between apposed body lumens or within a body vessel. Although FIG. 1D depicts two elongate tubular bodies disposed about the delivery tube, e.g., a first elongate tubular body 110a is disposed around the outer surface of a second tubular body 110b, in various embodiments three, four, five, six or more elongate tubular bodies may be disposed about the delivery tube. Various advantages may be realized by deploying multiple elongate tubular bodies of the present disclosure between apposed body lumens (e.g., in addition to enhanced flow provided by multiple drainage lumens). For example, fluid that might leak through the walls of the elongate tubular bodies does not pose a risk for body lumens that are in direct contact, e.g., in apposition, with each other. In addition to fluid flow through the lumen of each elongate tubular body, the slit 120 allows fluid to flow or “wick” along an outer surface of each elongate tubular body between the apposed body lumens. In addition, contact between the outer surfaces of multiple elongate tubular bodies may provide a de facto outer lumen along and through which additional fluid may flow.

In use, and by way of example, one or more elongate tubular bodies 110 may be pre-loaded on the delivery tube 130, or loaded by a physician immediately prior to performing a medical procedure. The delivery tube 130 loaded with the one or more elongate tubular bodies 110 may be advanced through the working channel of an echoendoscope. Once the physician has identified the fluid collection to be drained, and selected the desired access location, electrocautery or other access tip (not shown) may be activated and the delivery tube 130 advanced through the apposed tissue body lumen walls into the fluid collection. The elongate actuation member 140 may then be proximally retracted, while maintaining the position of the delivery tube 130, to laterally deploy the elongate tubular body (or bodies) from the distal portion 135 of the delivery tube 130. The retention members 124a, 124b may atraumatically contact or engage an inner portion (e.g., inner surface) of either or both body lumens, thereby limiting or preventing migration of the elongate tubular body 110. The delivery tube 130 and elongate actuation member 140 may then be proximally retracted to deploy, e.g., peel, the remaining portion of the elongate tubular body 110 (or bodies) from around the delivery tube 130. The delivery tube 130 may then be withdrawn from the patient, and proper stent placement confirmed by endoscopic and/or fluoroscopic imaging.

In one embodiment, an elongate tubular sheath (not shown) may be disposed around the delivery tube 130 and elongate tubular body 110 to maintain the retention members 124, 124b in a delivery configuration, e.g., lying flat against the outer surface of the elongate tubular body 110 and/or to maintain the position of the elongate tubular body on the delivery tube. The sheath may be proximally retracted prior to the deployment steps outlined above. Alternatively, a sheath may not be included, and the elongate tubular body 110 may be positioned on the delivery tube 130 such that a free end of the retention members 124a, 124b face proximally and are compressed as the delivery tube 130 is advanced through the echoendoscope working channel and through the opening formed between the apposed body lumens. In various embodiments, a pusher member may also be included about the delivery tube and proximal to the elongate tubular body or bodies to provide a backstop to the elongate body or bodies as the delivery tube is pulled proximally during deployment.

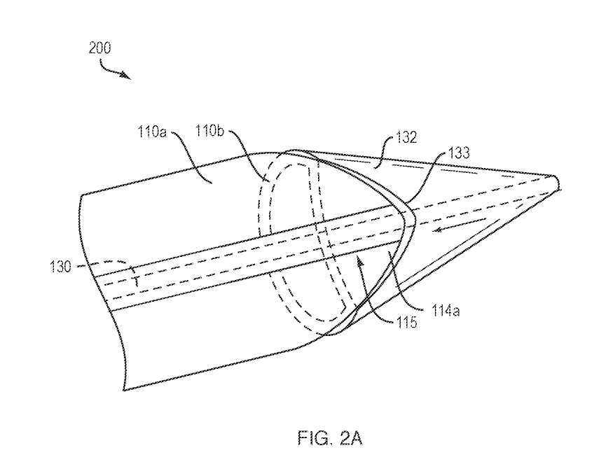

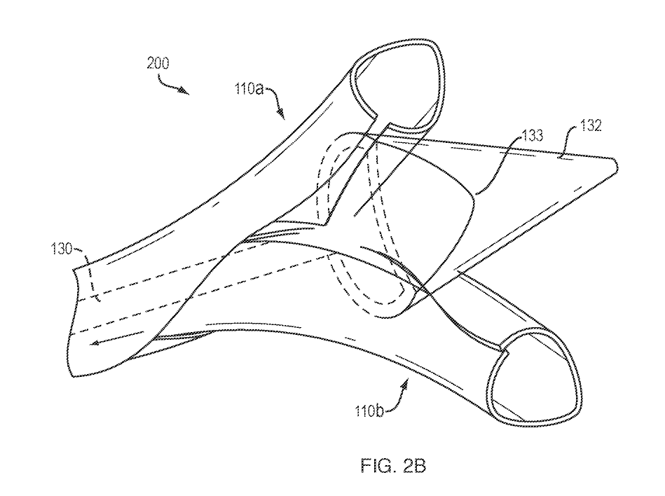

Referring to FIGS. 2A-2B, in one embodiment, a medical device 200 may include a delivery tube 130 similar to FIG. 1A, with the exception that the pair of slits 139a, 139b are replaced with a conical cap 132 attached to the distal end of the delivery tube 130. A distal end 114a of the first elongate tubular body 110a may include a chevron shape 115 configured to be received within a corresponding recess 133 formed within the conical cap 132 (FIG. 2A). As the delivery tube 130 is proximally retracted the distal end 114a of the first elongate tubular body 110a may ride up and over the recess 133 of the conical cap 132 to separate or peel the elongate tubular bodies 110a, 110b apart (FIG. 2B) from each other and from around the delivery tube.

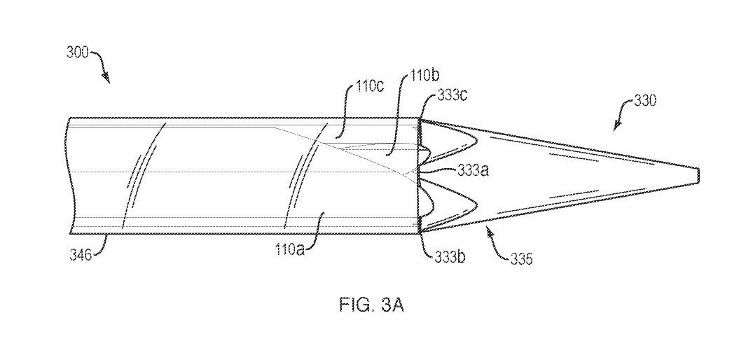

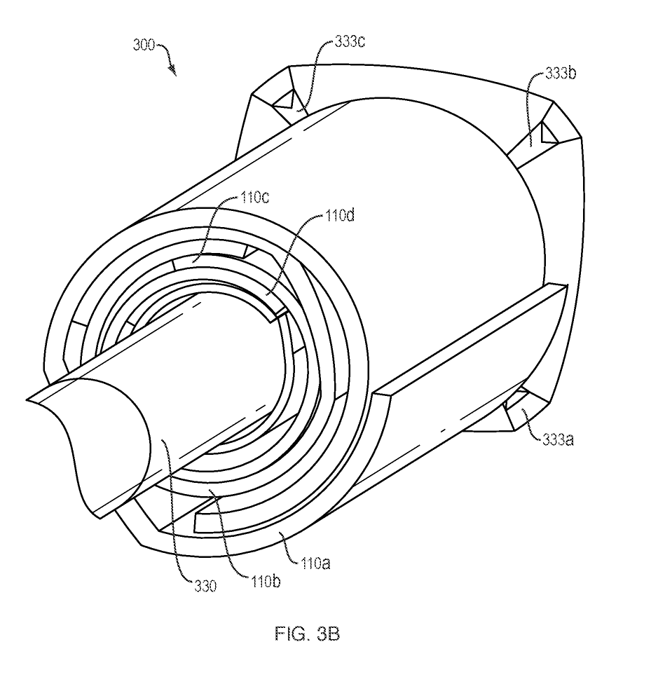

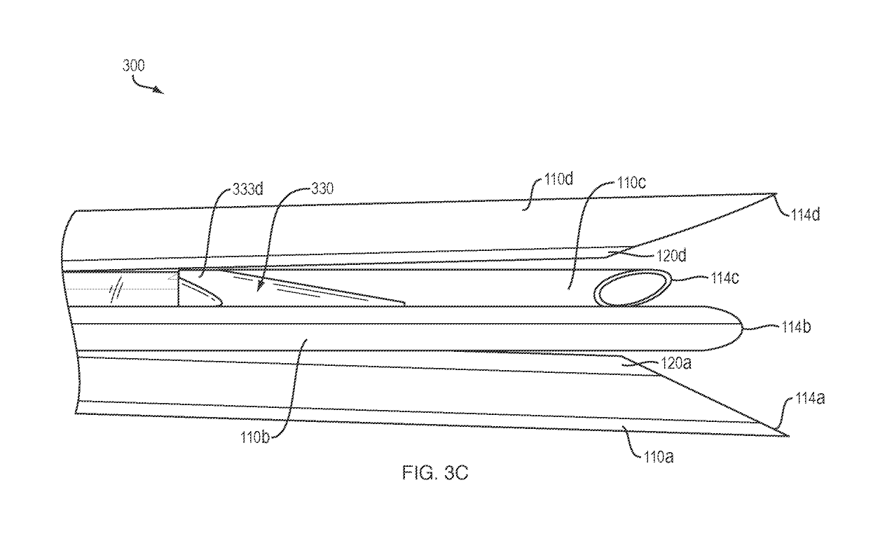

Referring to FIGS. 3A-3C, in one embodiment, a medical device 300 of the present disclosure may include four elongate tubular bodies 110a-d nested around the distal portion 335 of a delivery tube 330. A distal end of the delivery tube 330 may be attached to a conical cap 332. A distal portion of the conical cap may include a series of projections 333a-d, each of which may be configured to align with a corresponding slit 120a-d extending along a length of the respective elongate tubular body 110a-d. In one embodiment, the delivery tube 330 may be proximally retracted to draw each projection 120a-d through the corresponding slits 120a-d of the nested tubular bodies 110a-d, to simultaneously separate or peel each elongate tubular body 110a-d from around the delivery tube 330. In various embodiments, a distal end 114a-d of each elongate tubular body 110a-d may include a surface feature (e.g., protrusion, enlarged portion, etc.) configured to provide a space between (e.g., separate) the outer surfaces of each nested elongate tubular body 110a-d and guide projections 333a-d into the corresponding slits 120a-d. In one embodiment, an elongate tubular sheath 346 may be disposed around the outermost tubular body 110a, to maintain proper positioning of the elongate tubular bodies 110a-d on the delivery tube 330 and/or to maintain the retention members (not shown) in a delivery configuration, as discussed above. Although the medical device 200 depicted in FIGS. 3A-3C includes four elongate tubular bodies 110a-d, in various embodiments, any number of elongate tubular bodies (e.g., ten or more) may be disposed about the delivery tube 330. Again, with respect to FIGS. 2A-2B and 3A-3C, in various embodiments, a pusher member may also be included about the delivery tube and proximal to the elongate tubular body or bodies to provide a backstop to the elongate body or bodies as the delivery tube is pulled proximally during deployment.

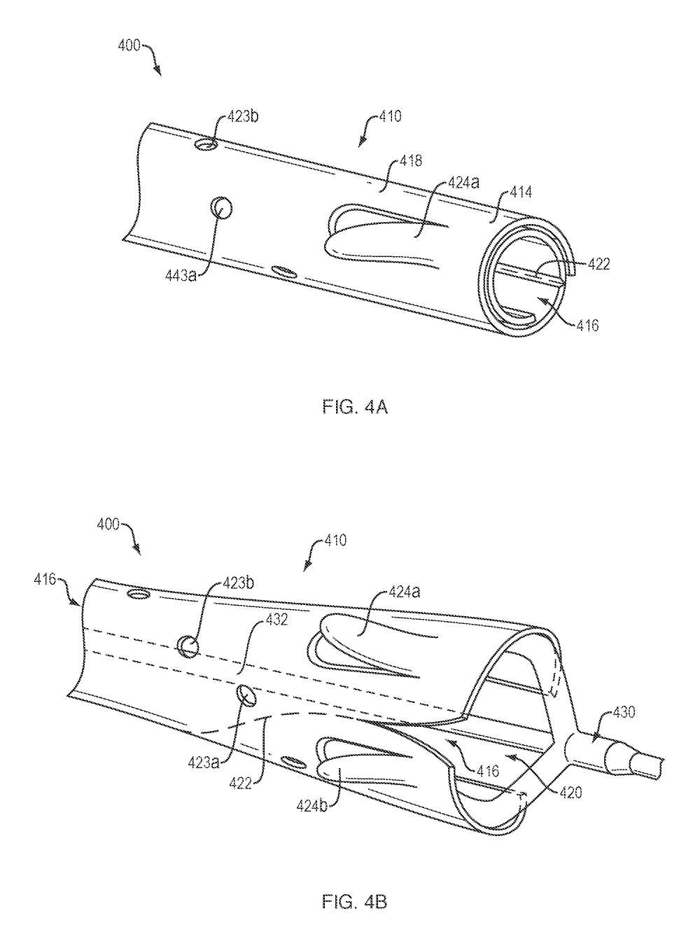

Referring to FIGS. 4A-4B, in one embodiment, a medical device 400 of the present disclosure may include an elongate tubular body 410 comprising a proximal end (not shown), a distal end 414 and a lumen 416 extending therebetween. A slit 420 may extend through a wall 418 of the elongate tubular body 410 along an entire length of one side of the elongate tubular body 410, and a perforation or score-line 422 may extend along an entire length of another side of the elongate tubular body 410 opposite the slit 420. One or more openings 423a, 423b may extend through the wall 418 of the elongate tubular body 410, and one or more retention members 424a, 424b may be disposed along a portion of the elongate tubular body 410. In one embodiment, the elongate tubular body 410 may include a compact geometry in which one edge of the elongate tubular body 410 defined by the slit 420 is radially coiled within the lumen 416 and the other edge of the elongate tubular body 410 defined by the slit 420 is disposed about an outer surface of the elongate tubular body 410.

In one embodiment, a dilation member 430 (e.g., balloon, malecot, structural spreader, etc.) may be positioned within the lumen 416 of the elongate tubular body 410 and moved from a first (e.g., non-expanded) configuration to a second (e.g., expanded) configuration with sufficient outward force to open the radially coiled elongate tubular body 410 and split the wall 418 along the score-line 422. In one embodiment, the dilation member 430 may be attached to the distal end of an elongate member 432 extending through the lumen 416 of the elongate tubular body 410. The elongate member 432 may be proximally retracted to draw the expanded dilation member 430 through the lumen 416 to separate the elongate tubular body 410 along the entire length of the score-line 422 to form two separate elongate tubular bodies (not shown). Alternatively, the medical device 400 may be loaded onto a delivery tube 130, e.g., as depicted in FIG. 1A, and deployed as discussed above. A pusher may optionally be used as a backstop as described above.

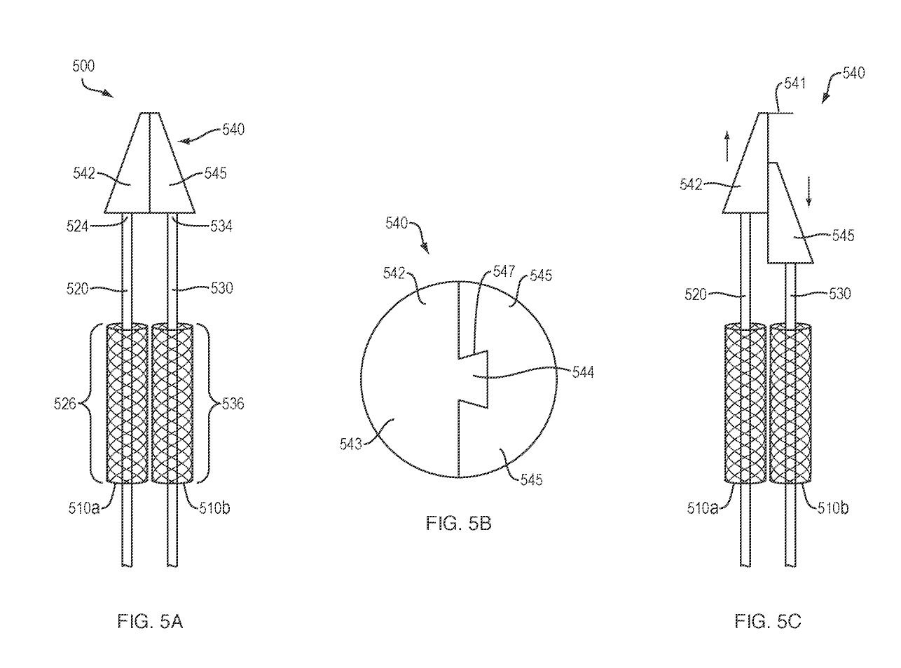

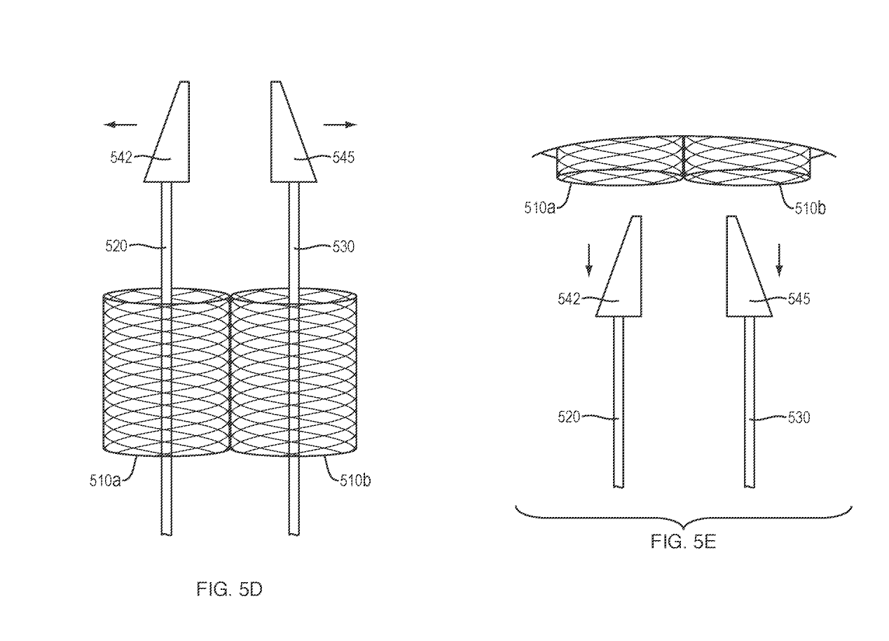

Referring to FIGS. 5A-5E, in one embodiment, a medical device 500 of the present disclosure may include a first elongate member 520 comprising a proximal end (not shown) and a distal end 524, and a second elongate member 530 comprising a proximal end (not shown) and a distal end 534. The distal end 524 of the first elongate member 520 may be attached to a first member 542 of a delivery tip 540 (e.g., electrocautery tip), and the distal end 534 of the second elongate member 530 may be attached to a second member 545 of the delivery tip 540. A first elongate tubular body 510a (e.g., a self-expanding metallic stent, etc.) may be disposed about a distal portion 526 of the first elongate member 520 in a radially constrained configuration, and a second elongate tubular body 510b (e.g., a self-expanding metallic stent, etc.) may be disposed about a distal portion 536 of the second elongate member 530 in a radially constrained configuration. A surface 543 of the first member 542 may include a ridge or protrusion 544 extending along a length thereof, and a surface 546 of the second member 545 may include a corresponding recess or groove 547 extending along a length thereof and configured to slidably receive the protrusion 544 (FIG. 5B), similar to a dovetail joint configuration. In one embodiment, the protrusion 544 and recess 547 may form a friction or interference fit with sufficient force to maintain the first and second members 542, 545 in a first configuration (FIG. 5A), e.g., for delivery through the working channel of an echoendoscope to form an opening between apposed body lumens, as discussed above. The friction or interference fit may be sufficiently weak to allow the first and second members 542, 545 to be separated from each other, e.g., by proximally retracting the first elongate member 520 and/or proximally advancing the second elongate member 530 (FIG. 5C), or vice versa, relative to the other. In various other embodiments, a portion of the surfaces 543, 546 of the first and second members 542, 545 may include a magnetic material with opposing polarities to maintain the first and second members 542, 545 in the first configuration, but sufficiently weak to allow the first and second members 542, 545 to be separated, as discussed above. In one embodiment, a distal end of the first member 542 may include an edge or stop 541 configured to contact a distal end of the second member 545, e.g., to prevent the second member 545 from moving/sliding distally beyond the first member 542.

In use, and by way of example, a medical device 500 loaded with the first and second elongate tubular bodies 510a, 510b may be advanced through the working channel of an echoendoscope. Once the physician has identified the fluid collection to be drained and selected the desired access location, the electrocautery delivery tip 540 may be activated and advanced through the tissue of the apposed body lumen walls into the fluid collection. The first and second members 542, 545 may then be disengaged as discussed above, thereby releasing the first and second elongate tubular bodies 510a, 510b from the constrained configuration (FIG. 5D). The first elongate member 520 and first member 542 may then be withdrawn through the lumen of the first elongate tubular body 510a, and the second elongate member 530 and second member 545 may be withdrawn through the lumen of the second elongate tubular body 510b. The first and second elongate members 520, 530 may then be withdrawn from the patient, and proper placement of the first and second elongate tubular bodies 510a, 510b confirmed by endoscopic and/or fluoroscopic imaging.

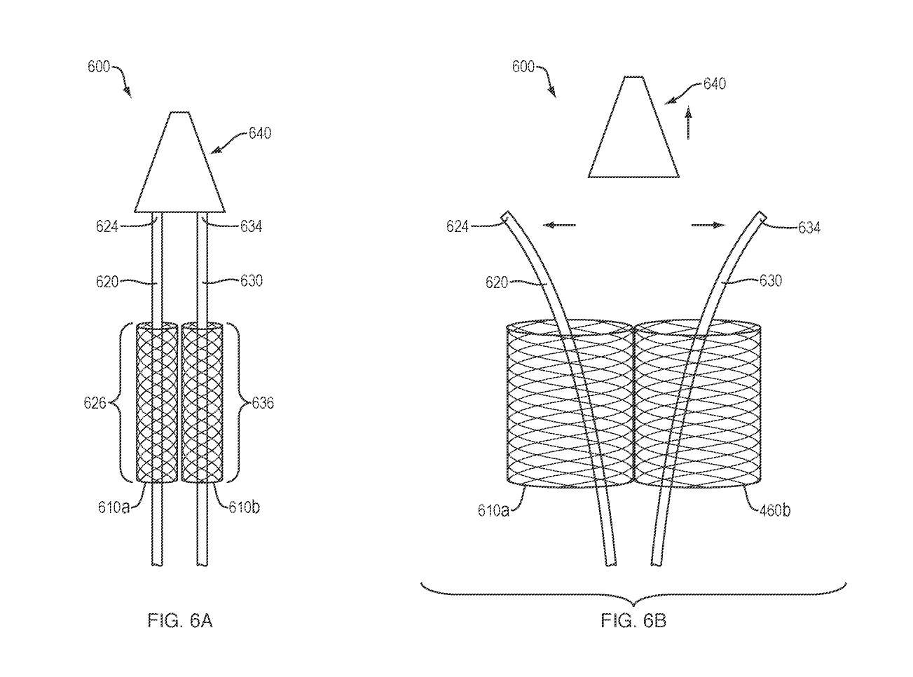

Referring to FIGS. 6A-6B, in one embodiment, a medical device 600 of the present disclosure may include a first elongate member 620 comprising a proximal end (not shown) and a distal end 624, and a second elongate member 630 comprising a proximal end (not shown) and a distal end 634. The distal ends 624, 634 of the first and second elongate members 620, 630 may be reversibly attached to a delivery tip 640 (e.g., electrocautery tip). For example, the distal ends 624, 634 may include a frangible, or electrically erodible, material configured to break, separate or degrade upon receiving an appropriate signal or retractive force, e.g., from the physician. A first expandable elongate tubular body 610a (e.g., a self-expanding metallic stent, etc.) may be disposed about a distal portion 626 of the first elongate member 620 in a constrained configuration, and a second elongate tubular body 610b (e.g., a self-expanding metallic stent, etc.) may be disposed about a distal portion 636 of the second elongate member 630 in a constrained configuration.

In use, and by way of example, a medical device 600 loaded with the first and second elongate tubular bodies 610a, 610b may be advanced through the working channel of an echoendoscope. Once the physician has identified the fluid collection to be drained and selected the desired access location, the electrocautery delivery tip 640 may be activated and advanced through the tissue of the apposed body lumen walls into the fluid collection. The delivery tip 640 may then be disengaged from first and second elongate members 620, 630, thereby releasing the first and second elongate tubular bodies 610a, 610b from the constrained configuration. The first elongate member 620 may then be withdrawn through the lumen of the first elongate tubular body 610a, and the second elongate member 630 may be withdrawn through the lumen of the second elongate tubular body 610b. The first and second elongate members 620, 630 may then be withdrawn from the patient, and proper placement of the elongate tubular bodies 610a, 610b confirmed by endoscopic and/or fluoroscopic imaging.

In one embodiment, the detached delivery tip 640 may remain within the second body lumen for removal during the body's natural course. Alternatively, the delivery tip 640 may remain attached to one of the first or second elongate members 620, 630 and removed through the respective expanded lumen of the first or second elongate tubular body 610a, 610b, as discussed above.

In various embodiments, the medical devices 500, 600 may further include an outer sheath (not shown) to maintain the elongate tubular bodies 510a, 510b, 610a, 610b in the constrained configuration on the respective distal portions 526, 626, 536, 636 of the first and second elongate members 520, 620, 530, 630. The sheath may be retracted prior to deploying the first and second elongate tubular bodies 510a, 510b, 610a, 610b. In various other embodiments, the first and second elongate members 520, 620, 530, 630 may include materials with sufficient columnar strength and flexibility (e.g., nitinol wire, etc.) to navigate through the working channel of the echoendoscope and through narrow and/or tortuous body lumens or passages.

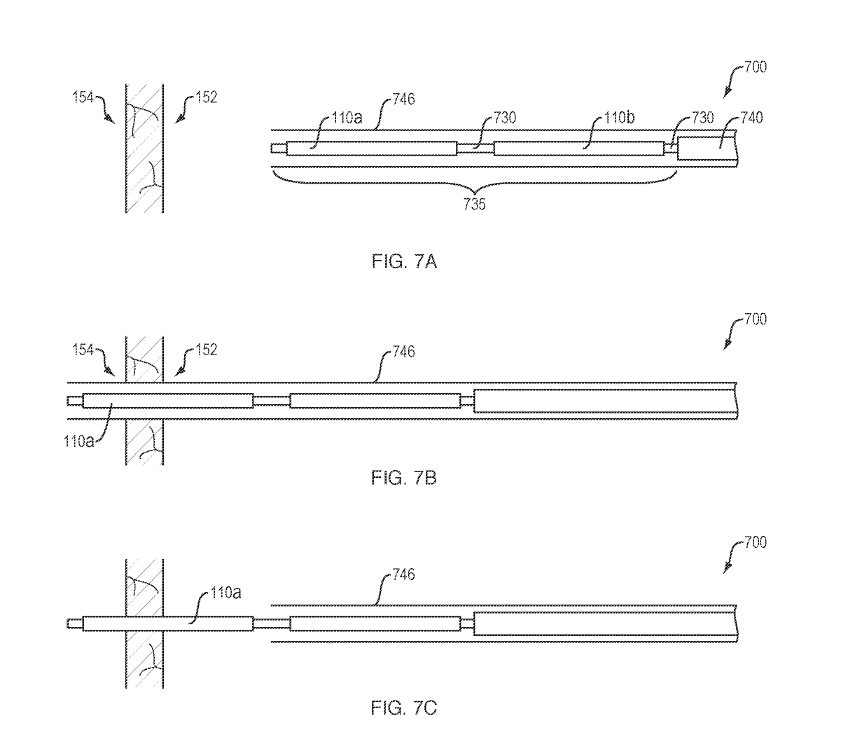

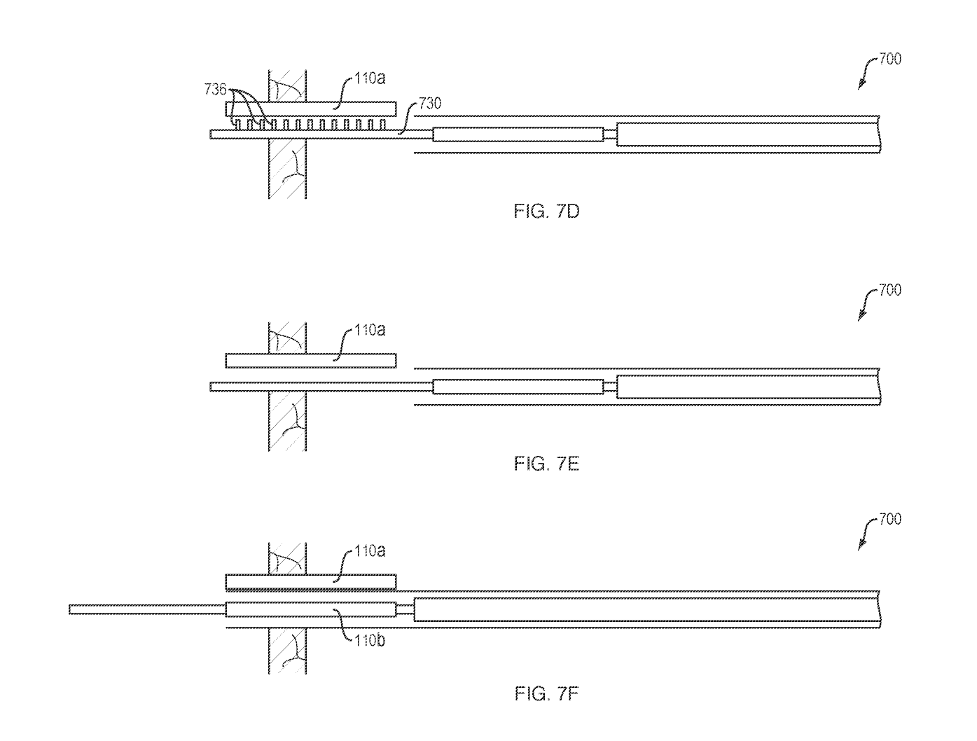

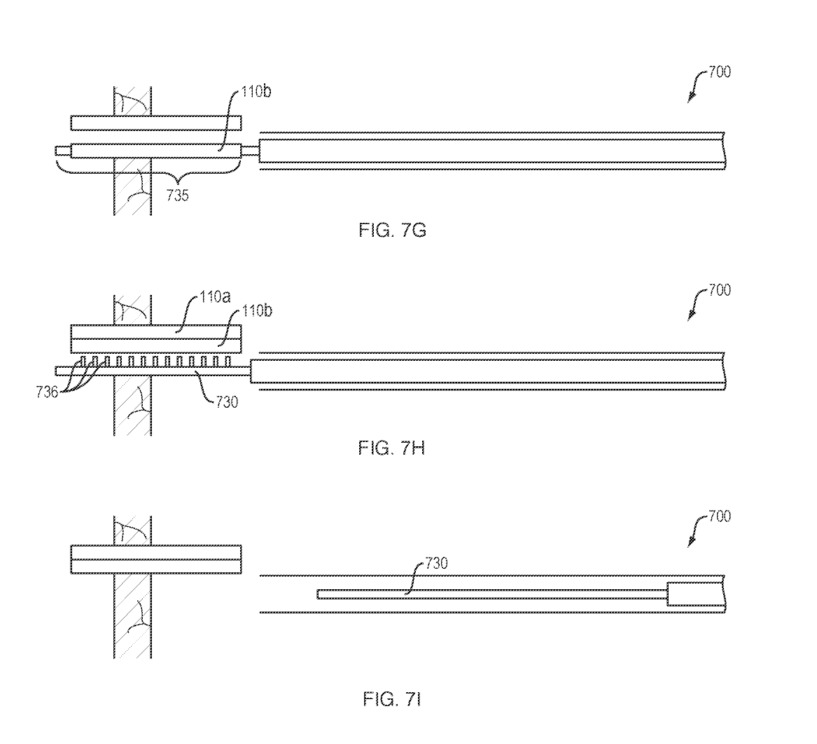

Referring to FIGS. 7A-7I, in one embodiment, a medical device 700 of the present disclosure may include a delivery tube 730, a sheath 746 and pusher member 740 configured to sequentially deliver two or more elongate tubular bodies 110a, 110b, disposed about the delivery tube, between apposed first and second body lumens 152, 154. For example, elongate tubular body 110a may be disposed around a distal portion of the delivery tube 730 and elongate tubular body 110b may be disposed around a proximal portion of the delivery tube 730. Each of the elongate tubular bodies 110a, 110b may include a longitudinal slit (not shown) extending along a length thereof, as discussed above. The sheath 746 may be slidably disposed about an outer surface of the delivery tube 730 and the pusher member 740. In one embodiment, a distal end of the pusher member 740 may be permanently attached or locked to a proximal end of the delivery tube 730. Alternatively, the pusher member 740 and delivery tube 730 may be locked and move in relation to each other, and unlocked (e.g., via a handle mechanism actuated by the physician) to move independently of each other. In various embodiments, the distal portion 735 of the delivery tube 730 may include one or more projections 736 (e.g., pegs, etc.) configured to move from a retracted configuration housed within the delivery tube 730, and an extended configuration in which the projections extend laterally outward from one side of the delivery tube 730 (FIG. 7D).

In use, and by way of example, the medical device 700 may be positioned at a desired location adjacent to a first body lumen 152 as discussed above, and advanced through the apposed first and second body lumens 152, 154 such that the distal most elongate tubular body 110a spans the apposed first and second body lumens (FIG. 7B). The sheath 746 may then be retracted to expose the distal most elongate tubular body 110a (FIG. 7C), and the pegs 736 moved to the extend configuration, e.g., via a handle mechanism activated by the physician, to laterally deploy the elongate tubular body 110a from around the delivery tube 730 (FIG. 7D) through the longitudinal slit (not shown). The pegs 736 may then be retracted into the delivery tube 730 (FIG. 7E). In one embodiment, to deploy the elongate tubular body 110b, the medical device 700 may be advanced until the proximal elongate tubular body 110b aligns with the deployed elongate tubular body 110a (FIG. 7F). The pusher member 740 and delivery tube 730 may then be unlocked, and the delivery tube 730 proximally retracted through the elongate tubular body 110b and pusher member 740, to position the distal portion 735 of the delivery tube 730 within the elongate tubular body 110b. The sheath 746 may then be retracted to expose the elongate tubular body 110b (FIG. 7G). The pegs 736 may then be moved to the extended configuration to laterally deploy the elongate tubular 110b off the delivery tube 730 adjacent to the previously deployed elongate tubular body 110a (FIG. 7H). Alternatively, after deploying the elongate tubular body 110a, the device may be maintained in position, and the pusher member 740 unlocked from the delivery tube 730 may push the elongate tubular body 110b distally along the delivery tube 730 until the elongate tubular body 110b is positioned over the pegs 736. The outer sheath may then be retracted and the pegs 736 extended to deploy elongate tubular body 110b, as described above. The pegs 736 may then be retracted into the delivery tube 730 and the medical device 700 removed from the patient (FIG. 7I). In various embodiments, the medical device 700 may be loaded with any number of elongate tubular bodies, and steps above repeated as necessary to sequentially deploy each elongate tubular body between the apposed first and second body lumens 152, 154.

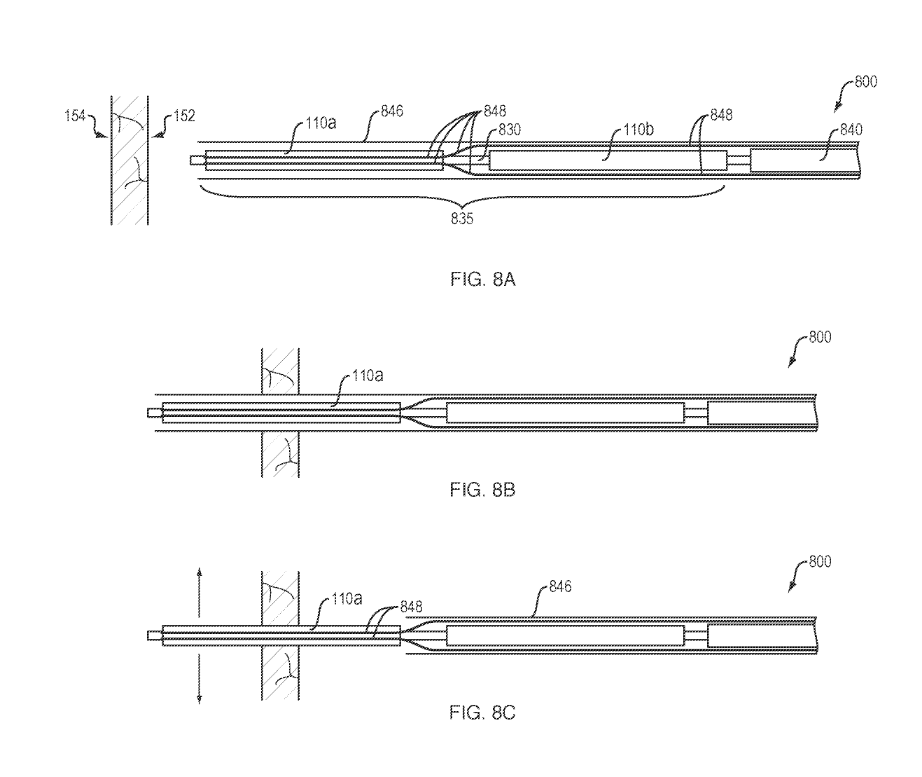

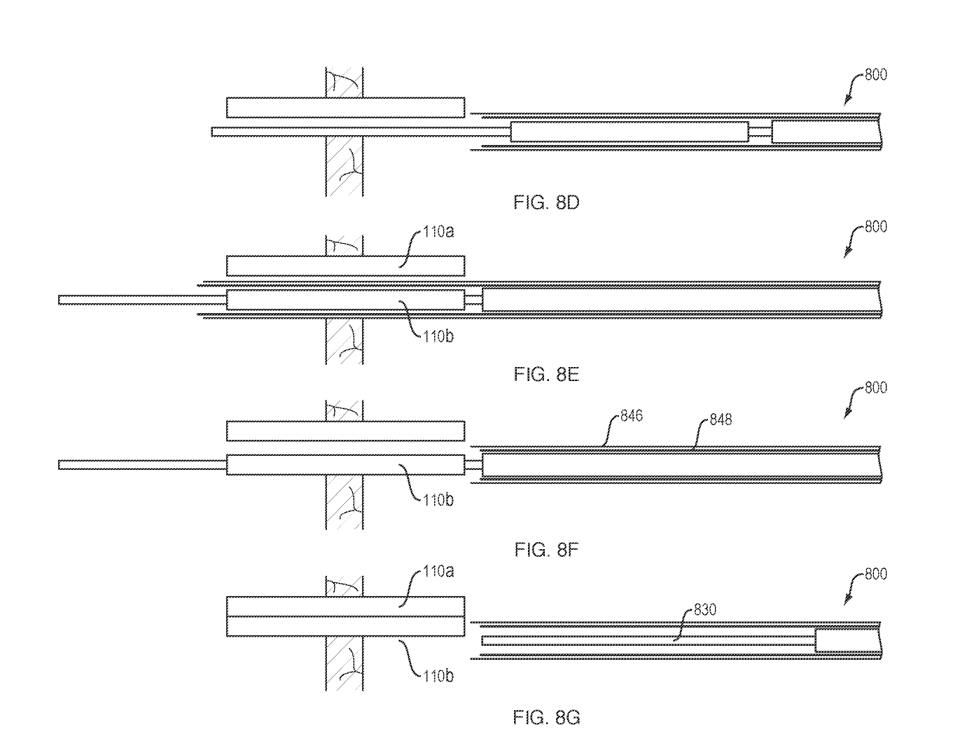

Referring to FIGS. 8A-8G, in one embodiment, a medical device 800 of the present disclosure may include a delivery tube 830, an outer sheath 846, an inner sheath 848 and pusher member 840 configured to sequentially deliver two or more elongate tubular bodies 110a, 110b disposed about a distal portion 835 of the delivery tube, between apposed first and second body lumens 152, 154. One or more shape memory wires (not shown) with a pre-disposed outward bias (e.g., toward the outer sheath 846) may be embedded within the inner sheath 848. A first portion of the inner sheath 848 may extend along an outer surface of the proximal most elongate tubular body 110b, and another portion of the inner sheath 848 may extend through the lumen of the distal most elongate tubular body 110a and along the delivery tube 830. The proximal most elongate tubular body 110a may include a longitudinal slit (not shown) extending along a length thereof, as discussed above.

In use, and by way of example, the medical device 800 may be positioned at a desired location adjacent to a first body lumen 152 as discussed above, and advanced through the apposed first and second body lumens 152, 154 such that the distal most elongate tubular body 110a spans the apposed first and second body lumens (FIG. 8B). The outer sheath 846 may then be retracted to expose the distal most elongate tubular body 110a (FIG. 8C) and release the shape memory wire(s) of the inner sheath 848 from constraint within the outer sheath 846 to laterally deploy the elongate tubular body 110a from around the delivery tube 830 through the longitudinal slit (FIG. 8C). In one embodiment, the inner sheath 848 may be proximally retracted to align with the outer sheath 846 (FIG. 8D). Alternatively, the inner sheath 848 may remain extended distally beyond the outer sheath (not shown). The medical device 800 may then be advanced until the proximal elongate tubular body 110b aligns with the deployed elongate tubular body 110a (FIG. 8E) and the inner and outer sheaths 848, 846 proximally retracted to expose the elongate tubular body 110b (FIG. 8F). The delivery tube 830 and/or medical device 800 may then be proximally retracted to remove the delivery tube 830 from within the lumen of the elongate tubular body 110b (FIG. 8G).

As exemplary methods, a pseudocyst drainage procedure may involve placing a medical device of the present disclosure between a pancreatic pseudocyst, walled-off pancreatic necrosis (WOPN) or pancreatic fluid collection (e.g., second body lumen) and the stomach or duodenum (e.g., first body lumen). A kidney drainage procedure may involve placing a medical device of the present disclosure between the kidney (e.g., second body lumen) and the bladder (e.g., first body lumen). A gallbladder or pancreatic drainage procedure may involve placing a medical device of the present disclosure within the common bile duct, pancreatic duct, common hepatic duct(s) and/or cystic duct.

In various embodiments, one or more retention members may be included in any of the elongate tubular bodies disclosed herein, and are not limited to configurations which engage the tissue walls of apposed first and second body lumens, but may also (or alternatively) engage a portion of the tissue wall of a body vessel to limit or prevent migration of the medical device. In addition, the retention members of the present disclosure are not limited to the shape depicted in FIGS. 1A-1B, but may include a variety of shapes, including, but not limited to, hooks, barbs, teeth, fingers, and/or projections with rounded or blunt edges or surfaces.

Although the medical devices 100, 200, 300, 400, 500, 600 of the present disclosure are described herein with respect to specific delivery systems, some of which may include an electrocautery tip (not shown), it should be appreciated that the various embodiments of the elongate tubular bodies disclosed herein may be configured to be loaded onto and delivered from the delivery wire of other examples of electrocautery stent delivery systems (e.g., Hot Axios™ Stent and Electrocautery-Enhanced Delivery System) immediately behind the electrocautery tip. A distal portion of the delivery wire may include an expandable portion (e.g., malecot) configured to laterally deploy the one or more elongate tubular bodies, as discussed above.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A medical device, comprising:
a delivery tube comprising:
a proximal end,
a distal end, and
a lumen extending between the proximal and distal ends;
an actuation member extending through the lumen of the delivery tube, the actuation member comprising a distal end attached to the distal end of the delivery tube; and
a first elongate tubular body disposed around a distal portion of the delivery tube, the first elongate tubular body comprising:
a proximal end,
a distal end,
a lumen extending between the proximal and distal ends, and
a slit extending along an entire length of the first elongate tubular body.

2. The medical device of claim 1, wherein the distal portion of the delivery tube is configured to move from a first position to an expanded second position when the actuation member is proximally retracted.

3. The medical device of claim 2, wherein a portion of the first elongate tubular body is deployable from around the distal portion of the delivery tube when the delivery tube moves from the first position to the second position.

4. The medical device of claim 2, wherein the distal portion of the delivery tube is extendable through the slit of the first elongate tubular body when in the second position.

5. The medical device of claim 2, wherein the distal portion of the delivery tube forms into a malecot configuration when in the second position.

6. The medical device of claim 1, further comprising a second elongate tubular body disposed around the first elongate tubular body, and the second elongate tubular body comprising a proximal end, a distal end, a lumen extending between the proximal and distal ends, and a slit extending along an entire length of the second elongate tubular body.

7. The medical device of claim 1, wherein the first elongate tubular body is a plastic stent.

8. A medical device, comprising:
a sheath;
a pusher member slidably disposed within a portion of the sheath;
a delivery tube slidably disposed within the sheath, wherein a proximal end of the delivery tube is attached to a distal end of the pusher member; and
a first elongate tubular body disposed about a distal portion of the delivery tube and a second elongate tubular body disposed about a proximal portion of the delivery tube, wherein each of the first and second elongate tubular bodies include a proximal end, a distal end and a slit extending along a length thereof.

9. The medical device of claim 8, further comprising one or more projections disposed within the distal portion of the delivery tube.

10. The medical device of claim 9, wherein the one or more projections are configured to move from a first position to a second position.

11. The medical device of claim 10, wherein the one or more projections are retractable within the distal portion of the delivery tube when in the first position.

12. The medical device of claim 10, wherein the one or more projections are extendable laterally outward from the distal portion of the delivery tube when in the second position.

13. The medical device of claim 12, wherein the one or more projections deploy the first elongate tubular body from about the distal portion of the delivery tube when in the second position.

14. The medical device of claim 13, wherein the pusher member is configured to advance the second elongate tubular body to the distal portion of the delivery tube after the first elongate tubular body is deployed.

15. The medical device of claim 14, wherein the one or more projections deploy the second elongate tubular body from about the distal portion of the delivery tube when in the second position.

16. A medical device, comprising:
an outer sheath;
a pusher member slidably disposed within a portion of the outer sheath;
a delivery tube slidably disposed within the outer sheath, wherein a proximal end of the delivery tube is attached to a distal end of the pusher member;
a first elongate tubular body disposed about a distal portion of the delivery tube and a second elongate tubular body disposed about a proximal portion of the delivery tube, wherein each of the first and second elongate tubular bodies includes a proximal end, a distal end and a slit extending along a length thereof; and
an inner sheath slidably disposed within the outer sheath, wherein a first portion of the inner sheath is disposed about the pusher member and the second elongate tubular body, and a second portion of the inner sheath is disposed within a lumen of the first elongate tubular body.

17. The medical device of claim 16, further comprising a shape memory wire embedded within the inner sheath.

18. The medical device of claim 16, wherein a portion of the inner sheath is configured to move between a first configuration and a second configuration to deploy the first elongate tubular body.

19. The medical device of claim 17, wherein the pusher member is configured to advance and deploy the second elongate tubular body.

20. The medical device of claim 16, wherein the inner sheath and outer sheath are retractable from the second elongate tubular body.
